(19) United States
(12) Patent Application Publication
(10) Pub. No.: US 2017/0196842 A1
(43) Pub. Date: Jul. 13, 2017

(54) METHODS OF TREATMENT USING A 1,2,4-OXADIAZOLE BENZOIC ACID

(71) Applicant: PTC Therapeutics, Inc., South Plainfield, NJ (US)

(72) Inventors: Marla L. WEETALL, Morristown, NJ (US); Ellen WELCH, Califon, NJ (US)

(21) Appl. No.: 15/316,415

(22) PCT Filed: Jun. 5, 2015
(86) PCT No.: PCT/US2015/034350

§ 371 Date: Dec. 5, 2016

Related U.S. Application Data
(60) Provisional application No. 62/009,100, filed on Jun. 6, 2014.

(51) Int. Cl. A61K 31/4245

(52) U.S. Cl. CPC A61K 31/4245

(57) ABSTRACT

Provided herein are methods of treating, preventing, ameliorating or managing mucopolysaccharidosis (nmMPS) disease, comprising administering a 1,2,4-oxadiazole benzoic acid to a patient having a nmMPS. In particular, provided herein are methods of treating, preventing, ameliorating or managing a MPS.

This application claims the benefit of priority to U.S. Provisional Application Ser. No. 62/009,100, filed Jun. 6, 2014, which is incorporated herein by reference in its entirety and for all purposes.

### 1. FIELD

Provided herein are methods of treating, preventing, ameliorating or managing a nonsense mutation mediated mucopolysaccharidosis (nmMPS) disease, comprising administering a 1,2,4-oxadiazole benzoic acid to a patient having a nmMPS. In particular, provided herein are methods of treating, preventing, ameliorating or managing a nmMPS type I disease (e.g., a nmMPS I: a nonsense mutation mediated Hurler syndrome, a nonsense mutation mediated Hurler-Scheie syndrome or a nonsense mutation mediated Scheie syndrome) associated with a nonsense mutation or a premature stop codon, comprising administering a 1,2,4-oxadiazole benzoic acid to a patient having nmMPS I associated with a nonsense mutation or a premature stop codon.

### 2. BACKGROUND

Nonsense mutation(s) in the gene producing α-L-iduronidase result in a nonsense mutation mediated mucopolysaccharidosis disease having a range of severity including, but not limited to, nonsense mutation mediated Hurler syndrome: MPS IH, nonsense mutation mediated Hurler-Scheie syndrome: MPS IH/S or nonsense mutation mediated Scheie syndrome: MPS IS (formerly MPS V). The presence of one or more nonsense mutation(s) may further result in a spectrum of nonsense mutation mediated mucopolysaccharidosis diseases, including those with one or more nonsense mutations in the gene producing the protein: i). iduronate sulfatase, resulting in Hunter syndrome (MPS II); ii). heparan sulfamidase, resulting in Sanfilippo syndrome A (MPS IIIA, also referred to as Sulfamidase deficiency); iii). N-acetylglucosaminidase, resulting in Sanfilippo syndrome B (MPS IIIB, also referred to as NAGLU deficiency); iv). heparin-α-glucosaminide-N-acetyltransferase, resulting in Sanfilippo syndrome C (MPS IIIC); v). N-acetylglucosamine-6-sulfatase, resulting in Sanfilippo syndrome D (MPS IIID); vi). galactose-6-sulfate-sulfatase, resulting in Morquio syndrome A (MPS IVA); vii). β-galactosidase, resulting in Morquio syndrome B (MPS IVB); viii). N-acetylgalactosamine-4-sulfatase, resulting in Maroteaux-Lamy syndrome A (MPS VI, also referred to as ARSB deficiency); ix). β-glucuronidase, resulting in Sly syndrome (MPS VII, also referred to as GUSB deficiency); or, x). hyaluronidase, resulting in Natowicz syndrome: MPS IX (also referred to as Hyaluronidase deficiency).

In particular, nonsense mutation mediated Mucopolysaccharidosis Type I (i.e., nmMPS I: a nonsense mutation mediated Hurler syndrome, a nonsense mutation mediated Hurler-Scheie syndrome or a nonsense mutation mediated Scheie Syndrome) is a chronic, progressive, multisystem lysosomal storage disease caused by a deficiency or a lack of activity of the α-L-iduronidase (IDUA) enzyme (EC3.2.1.76) (Campos D, Monaga M. Mucopolysaccharidosis type I: current knowledge on its pathophysiological mechanisms. Metabolic Brain Disease. 2012; 27(2):121-129). nmMPS I has an autosomal recessive inheritance pattern (Muenzer J, Wraith J E, Clarke L A, International Consensus Panel on M, Treatment of Mucopolysaccharidosis I. Mucopolysaccharidosis I: management and treatment guidelines. Pediatrics. 2009; 123(1):19-29). Mucopolysaccharide symptoms appear shortly after birth and the lack of IDUA results in the accumulation of glycosaminoglycans (GAGs), heparan sulphate (HS) and dermatan sulphate (DS) throughout the body causing widespread cellular, tissue, and organ dysfunction. Children with nmMPS I generally have an average life-span of 11.6 years, and have severe physical and cognitive disabilities (Moore D, Connock M J, Wraith E, Lavery C. The prevalence of and survival in Mucopolysaccharidosis I: Hurler, Hurler-Scheie and Scheie syndromes in the UK. Orphanet journal of rare diseases. 2008; 3:24). nmMPS I has two main classifications: attenuated form and severe form, with varying symptoms in each form (Muenzer J, Wraith J E, Clarke L A, International Consensus Panel on M, Treatment of Mucopolysaccharidosis I. Mucopolysaccharidosis I: management and treatment guidelines. Pediatrics. 2009; 123(1):19-29).

### 3. SUMMARY OF THE DISCLOSURE

Provided herein are methods of treating, preventing, ameliorating or managing a nonsense mutation mediated mucopolysaccharidosis (nmMPS) disease, comprising administering a 1,2,4-oxadiazole benzoic acid to a patient having a nmMPS disease. In particular, provided herein are methods of treating, preventing, ameliorating or managing a nonsense mutation mediated MPS Type I (nmMPS I: a nonsense mutation mediated Hurler syndrome, a nonsense mutation mediated Hurler-Scheie syndrome or a nonsense mutation mediated Scheie syndrome) associated with a nonsense mutation or a premature stop codon, comprising administering a 1,2,4-oxadiazole benzoic acid to a patient having nmMPS I associated with a nonsense mutation or a premature stop codon.

### 4. BRIEF DESCRIPTION OF THE DRAWINGS

FIGS. 1 (A and B) indicates that Compound 1 reduced tissue glycosaminoglycan (GAG) and dose-response in mouse embryonic fibroblasts (MEFs) isolated from homozygous nonsense mutation mice referred to as Idua-W402X (−/−) or Idua-W392X (−/−) mice. MEFs were treated with Compound 1 (1, 2.5, 5, 10, 20 and 40 μg/mL) for 24 hours. Controls included MEFs from wild-type mice (+/+) and untreated MEFs from Idua-W402X (−/−) MEFs. FIG. 1 (A) depicts the absolute GAG levels. FIG. 1 (B) depicts the percent reduction in excess tissue GAGs in Idua-W402X (−/−) MEFs which was calculated by first subtracting the background GAG levels as measured in MEFs from wild-type mice and then calculating the percent reduction in GAGs as compared to the untreated homozygous MEF control. SD refers to standard deviation.

FIG. 2 depicts Compound 1 plasma concentrations with increasing doses from 0.1% to 1.0% by weight. Symbols represent the average of 4 mice±standard error of the mean. Cₚ refers to plasma concentration and SEM refers to Standard Error of the Mean.

FIG. 3 depicts total protein concentrations of cells remaining stable after treatment with Compound 1. Wild-type (+/+) and mutant (−/−) MEFs were treated for 24 hr with Compound 1 concentrations ranging from 0 to 40 ug/mL and the total protein concentration was determined.

FIG. 4 depicts the effect of Compound 1 on GAG levels in MEFs isolated from homozygous Idua-W392X (−/−) mice. Cells were isolated and grown in the presence of Compound 1 for 24 hours. After the treatment, cells were collected and GAG levels and total protein levels were determined. The results are presented as ng GAG/mg total protein. All experiments were performed in triplicate and the average value was used for the graphical presentation. Error bars represent standard deviation. For reference, the GAG level determined from wild type (+/+) cells is marked by +/+.

FIG. 5 (A-F) depicts the effect of Compound 1 on GAG levels. Tissues were isolated and GAG levels and total protein levels were determined. The results are presented as GAG/mg total protein (ng). Error bars represent standard deviation. FIG. 5 (A) brain, FIG. 5 (B) spleen, FIG. 5 (C) heart, FIG. 5 (D) lung, FIG. 5 (E) liver, FIG. 5 (F) kidney. +/+: wild type or −/−: Idua-W392X. Statistical significance was determined by using the Student's t-test.

### 5. DETAILED DESCRIPTION

### 5.1. Definitions

As used herein, the term “premature translation termination” refers to the result of a mutation that changes a codon corresponding to an amino acid to a stop codon.

As used herein, the term “nonsense-mediated mRNA decay” refers to any mechanism that mediates the decay of mRNAs containing a premature translation termination codon. In one embodiment, the nonsense-mediated mRNA decay results from a nonsense mutation of DNA.

As used herein, the term “premature termination codon” or “premature stop codon” refers to the occurrence of a stop codon where a codon corresponding to an amino acid should be.

As used herein, the term “nonsense mutation” refers to a mutation changing a codon corresponding to an amino acid to a stop codon. In one embodiment, the nonsense mutation is a mutation that occurs in DNA and is then transcribed into mRNA.

As used herein, the term “nonsense suppression” refers to the inhibition or suppression of premature translation termination and/or nonsense-mediated mRNA decay. In one embodiment, the mRNA decay results from a nonsense mutation of DNA.

As used herein, the term “modulation of premature translation termination and/or nonsense-mediated mRNA decay” refers to the upregulation of gene expression in the presence of a nonsense suppression agent. For example, if it is desirable to increase production of a defective protein encoded by a gene with a premature stop codon, i.e., to permit read through of the premature stop codon of the disease gene so translation of the mRNA can occur, then modulation of premature translation termination and/or nonsense-mediated mRNA decay requires the use of a nonsense suppression agent.

As used herein, the terms “active agent,” “drug,” and “drug substance” refer to 3-[5-(2-fluoro-phenyl)-[1,2,4]oxadiazol-3-yl]-benzoic acid or a pharmaceutically acceptable salt thereof provided herein (collectively referred to herein as “Compound 1”).

As used herein, the term “dose(s)” means a quantity of active agent to be administered at one time.

As used herein, the term “unit dosage form(s)” includes solid dosage forms such as tablets, caplets, capsules, lozenges, dispersions, powders, granules or gels and the like or liquid dosage forms such as solutions, suspensions, emulsions or elixirs and the like and solid forms that can be reconstituted to provide such liquid dosage forms, wherein such unit dosage form(s) are suitable for oral or parenteral administration to a patient.

As used herein, the terms “dosing regimen” and “dosage(s)” mean the amount of an active agent given per time unit and the duration of administration.

As used herein, the terms “subject” and “patient” are used interchangeably to refer to an animal or any living organism having sensation and the power of voluntary movement, and which requires for its existence oxygen and organic food. Non-limiting examples include members of the human, primate, equine, porcine, bovine, leporine, rattus, murine, canine and feline species. In some embodiments, the subject is a mammal or a warm-blooded vertebrate animal. In certain embodiments, the subject is a non-human animal. In specific embodiments, the subject is a human. In certain embodiments, the subject is a fetus, embryo, infant, child, adolescent or adult. In one embodiment, it has been determined through pre-screening that the subject possesses a nonsense mutation. In another embodiment, it has been determined through genetic pre-screening which premature stop codon the patient has (i.e., UAA, UGA, or UAG).

As used herein, the term “effective amount” in the context of a functional read-through protein refers to the amount of the functional read-through protein(s) that has a prophylactic and/or therapeutic benefit to a subject. In specific embodiments, an effective amount of a functional read-through protein is the amount of protein that has in one, two or more of the following effects: (1) prevent the onset, development and/or progression of a nmMPS disease associated with a nonsense mutation(s), prevent the onset, development and/or progression of one or more symptoms associated with a nmMPS disease associated with a nonsense mutation(s), (3) reduce the duration and/or severity of a nmMPS disease associated with a nonsense mutation(s), (4) reduce the number of symptoms associated with a nmMPS disease associated with a nonsense mutation(s), (5) reduce the duration of one or more symptoms associated with a nmMPS disease associated with a nonsense mutation(s), (6) reduce the severity of one or more symptoms associated with a nmMPS disease associated with a nonsense mutation(s) and (7) improve the quality of life of a subject. Symptoms of a nmMPS disease include cognitive impairment, enlarged tongue, hearing loss, developmental delays, communicating hydrocephalus, sinus symptoms (including sinus infections in the attenuated form, and chronic recurrent rhinitis, nasal discharge, and ear infections in the severe form), hearing loss (including less severe in the attenuated patients), impaired vision (including corneal clouding and other vision symptoms including loss of peripheral vision and night blindness), respiratory manifestations, cardiac manifestations, skeletal manifestations, dental manifestations, severe respiratory insufficiencies due to restrictive lung disease, severe sleep apnoea, asthma, enlarged tonsils, enlarged adenoids, narrowed trachea, excess airway tissue, thickened vocal cords, valvular disease, arrhythmia, cardiomyopathy, congestive heart failure, coronary artery disease, pulmonary and systemic hypertension, cor pulmonale, gum abnormalities, tooth abnormalities, enamel abnormalities, frequent caries, dentigerous cysts, and abscesses.

As used herein, the term “effective amount” in the context of the administration of a compound described herein refers to the amount of the compound that has a prophylactic and/or therapeutic benefit to a subject. In specific embodiments, an effective amount of a compound described herein that has in one, two or more of the following effects: (1) prevents the onset, development and/or progression of a nmMPS disease associated with a nonsense mutation(s), (2) prevents the onset, development and/or progression of one or more symptoms associated with a nmMPS disease associated with a nonsense mutation(s), (3) reduces the duration and/or severity of a nmMPS disease associated with a nonsense mutation(s), (4) reduces the number of symptoms associated with a nmMPS disease associated with a nonsense mutation(s), (5) reduces the duration of one or more symptoms associated with a nmMPS disease associated with a nonsense mutation(s), (6) reduces the severity of one or more symptoms associated with a nmMPS disease associated with a nonsense mutation(s) and/or (7) improves the quality of life of a subject. Examples of effective amounts of a compound described herein are provided in Section 5.4, infra.

As used herein, the term “functional” in the context of a functional read-through protein refers to a protein that has enough of the functions of the corresponding wild-type protein to have a beneficial effect in a cell or subject which does not produce or produces insufficient amounts of the wild-type protein as a result of a mutation (e.g., a nonsense mutation) in the nucleic acid sequence (e.g., gene) encoding the protein. In a specific embodiment, the functional read-through protein(s) has one, two, three or more functions of the full-length wild-type protein(s). In certain embodiments, the functional read-through protein(s) produced is a functional non-wild-type protein(s). In certain embodiments, the functional read-through protein(s) produced is a functional wild-type protein(s). In some embodiments, the functional non-wild-type protein produced is full-length. In some embodiments, the functional wild-type protein produced is full-length. In other embodiments, the functional non-wild-type protein(s) is not full-length. In other embodiments, the functional wild-type protein(s) produced is not full-length.

As used herein, the term “a nmMPS disease” refers to a disease or condition resulting either directly or indirectly from a nonsense mutation(s) in a gene(s), where the nonsense mutation(s) prevents production of a wild-type protein in an affected cell. In a specific embodiment, for nmMPS I, the gene is the IDUA gene. MPS I associated with a nonsense mutation in the IDUA gene encompasses diseases in which the gene contains one, two, three or more nonsense mutations. In a specific embodiment, the nonsense mutation(s) in the gene producing α-L-iduronidase results in a nonsense mutation mediated mucopolysaccharidosis disease having a range of severity including, but not limited to, nonsense mutation mediated Hurler syndrome: nmMPS IH, nonsense mutation mediated Hurler-Scheie syndrome: nmMPS IH/S or nonsense mutation mediated Scheie syndrome: nmMPS IS (formerly MPS V). In other embodiments, the nonsense mutation(s) is in two, three or more (multiple) genes (e.g., genes that contain one, two, three or more nonsense mutations resulting in a spectrum of mucopolysaccharidoses. In such a specific embodiment, the nonsense mutation(s) in the gene producing iduronate sulfatase results in nonsense mutation mediated Hunter syndrome: nmMPS II. In another specific embodiment, the nonsense mutation(s) in the gene producing heparan sulfamidase results in nonsense mutation mediated Sanfilippo syndrome A: nmMPS IIIA (also referred to as Sulfamidase deficiency). In another specific embodiment, the nonsense mutation(s) in the gene producing N-acetylglucosaminidase results in nonsense mutation mediated Sanfilippo syndrome B: nmMPS IIIB (also referred to as NAGLU deficiency). In another specific embodiment, the nonsense mutation(s) in the gene producing heparin-α-glucosaminide-N-acetyltransferase results in nonsense mutation mediated Sanfilippo syndrome C: nmMPS IIIC. In another specific embodiment, the nonsense mutation(s) in the gene producing N-acetylglucosamine-6-sulfatase results in nonsense mutation mediated Sanfilippo syndrome D: nmMPS IIID. In another specific embodiment, the nonsense mutation(s) in the gene producing galactose-6-sulfate-sulfatase results in nonsense mutation mediated Morquio syndrome A: nmMPS IVA. In another specific embodiment, the nonsense mutation(s) in the gene producing β-galactosidase results in nonsense mutation mediated Morquio syndrome B: nmMPS IVB. In another specific embodiment, the nonsense mutation(s) in the gene producing N-acetylgalactosamine-4-sulfatase results in nonsense mutation mediated Maroteaux-Lamy syndrome A: nmMPS VI (also referred to as ARSB deficiency). In another specific embodiment, the nonsense mutation(s) in the gene producing β-glucuronidase results in nonsense mutation mediated Sly syndrome: nmMPS VII (also referred to as GUSB deficiency). In another specific embodiment, the nonsense mutation(s) in the gene producing hyaluronidase results in nonsense mutation mediated Natowicz syndrome: nmMPS IX (also referred to as Hyaluronidase deficiency).

As used herein, “in combination” in the context of the administration of therapies refers to the use of more than one therapy. The use of the term “in combination” does not restrict the order in which therapies are administered to a subject with a disease. In certain embodiments, administration of one or more therapies to a subject with a disease includes, without limitation, a first therapy that can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to a disease. The therapies are administered to a subject in a sequence and within a time interval such that a unit dosage form(s) described herein can act together with another therapy to provide an increased benefit than if the therapies were administered alone.

As used herein, the terms “manage,” “managing” and “management” refer to the beneficial effects that a patient derives from the administration of a pharmaceutical composition provided herein comprising 3-[5-(2-fluoro-phenyl)-[1,2,4]oxadiazol-3-yl]benzoic acid or a salt thereof, which does not result in treating, preventing or ameliorating the nonsense mutation mediated disease.

As used herein, the terms “prevent,” “preventing” and “prevention” refer to the prevention of the onset, recurrence, spread or worsening of the nonsense mutation mediated disease or a symptom thereof (and thus treat or, at least, ameliorate such disease) in a patient from the administration of a pharmaceutical composition provided herein comprising administering 3-[5-(2-fluoro-phenyl)-[1,2,4]oxadiazol-3-yl]benzoic acid or a salt thereof to a patient with such a disease. Since diseases associated with a nonsense mutation have a genetic basis, a patient can be screened for the presence of a nonsense mutation. When it is determined through screening that a patient has a nonsense mutation, an effective amount of a pharmaceutical composition comprising an effective amount of 3-[5-(2-fluoro-phenyl)-[1,2,4]oxadiazol-3-yl]benzoic acid or a salt thereof provided herein can be administered to the patient to prevent the onset, recurrence, spread or worsening of the disease or a symptom thereof.

As used herein, the terms “treat,” “treating” and “treatment” refer to the eradication or amelioration of the disease or symptoms associated with the disease. In certain embodiments, such terms refer to minimizing the spread or worsening of the disease in a patient from the administration of a pharmaceutical composition provided herein comprising 3-[5-(2-fluoro-phenyl)-[1,2,4]oxadiazol-3-yl]benzoic acid or a salt thereof provided herein to a patient with such a disease. When it is determined that a patient has a disease associated with a nonsense mutation, an effective amount of a pharmaceutical composition comprising an effective amount of 3-[5-(2-fluoro-phenyl)-[1,2,4]oxadiazol-3-yl]benzoic acid or a salt thereof provided herein can be administered to the patient to eradicate, ameliorate, minimize the spread or the worsening of the disease or a symptom thereof.

As used herein, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

### 5.2. The Compound

A compound for use in the preparation of the pharmaceutical compositions and salts provided herein is 3-[5-(2-fluoro-phenyl)-[1,2,4]oxadiazol-3-yl]-benzoic acid, also generically referred to as ataluren, having the structure of Formula (I):

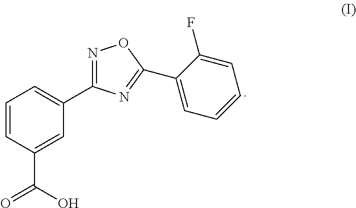 [SMILES: O=C(O)c1cccc(-c2noc(-c3ccccc3F)n2)c1]

A compound of Formula (I) can be prepared according to the methods described in U.S. Pat. No. 6,992,096 and U.S. Pat. No. 7,678,922, the disclosure of each of which is incorporated by reference herein in its entirety. Alternatively, a compound of Formula (I) can be also prepared according to other methods apparent to those of skill in the art based upon the teaching herein. All such alternative methods are intended to be included within the scope of the methods described herein. The compound of Formula (I) and salts provided herein are collectively referred to as “Compound 1.”

In one embodiment, Compound 1 used in the pharmaceutical compositions, processes, and methods provided herein is a free acid. In one embodiment, the free acid is a solid. In yet another embodiment, the solid free acid is a crystalline form described in U.S. Pat. No. 7,863,456, the disclosure of which is incorporated by reference herein in its entirety. In yet another embodiment, the solid free acid is a crystalline Form A. In yet another embodiment, the solid free acid is a crystalline Form B. These solid forms of the compound of Formula (I) can also be prepared according to the methods described in U.S. Pat. No. 7,863,456, the disclosure of which is incorporated by reference herein in its entirety. Alternatively, the solid forms of the compound of Formula (I) can be also prepared according to other methods apparent to those of skill in the art based upon the teaching herein.

In another embodiment, the free acid of the compound of Formula (I) is a pharmaceutically acceptable solvate. In one embodiment, the free acid is a hydrate. In another embodiment, the compound of Formula (I) is a pharmaceutically acceptable anhydrous form. In another embodiment, the free acid of the compound of Formula (I) is a pharmaceutically acceptable cocrystal form such as a clathrate or a complex with a cyclodextrin.

In another embodiment, Compound 1 used in the pharmaceutical compositions, processes, and methods provided herein is a pharmaceutically acceptable free acid of the compound of Formula (I). In another embodiment, Compound 1 used in the pharmaceutical compositions, processes, and methods provided herein is a pharmaceutically acceptable salt of the compound of Formula (I). In another embodiment, Compound 1 used in the pharmaceutical compositions, processes, and methods provided herein is a pharmaceutically acceptable anhydrous free acid or salt of the compound of Formula (I).

### 5.3. Salt Forms

In certain embodiments, the methods provided herein comprise the use of salt forms of Compound 1, including salts selected from L-arginine, L-histidine, L-lysine, N-methyl glucamine, magnesium methoxide, potassium hydroxide, sodium hydroxide or tromethamine (PCT Application No. PCT/US2015/18889, filed Mar. 5, 2015, which is incorporated by reference herein in its entirety). More particularly, the methods provided herein comprise the use of salt forms of Compound 1 selected from L-lysine, sodium hydroxide and tromethamine.

### 5.4. Pharmaceutical Compositions

Pharmaceutical compositions and single unit dosage forms comprising an effective amount of Compound 1 can be used in the methods provided herein. Individual dosage forms may be suitable for oral, dermal, mucosal (including, without limitation, sublingual, buccal, rectal, nasal, or vaginal) or parenteral (including, without limitation, subcutaneous, intramuscular, intraarterial, intraperitoneal, intrathecal, intraventricular, intraurethral, intrasternal, intracranial, intrasynovial, intravesical or intravenous) or ocular administration. Preferred pharmaceutical compositions and single unit dosage forms are suitable for oral administration.

In certain embodiments, the pharmaceutical composition comprises from about 0.1% to about 99%, from about 5% to about 90%, from about 5% to about 50%, from about 10% to about 40%, from about 20% to about 30%, from about 0.1% to about 5%, from about 0.1% to about 2.5%, from about 0.1% to about 1% or from about 0.25% to about 0.5% by weight of Compound 1. In certain embodiments, the pharmaceutical composition comprises about 0.1%, about 0.25%, about 0.5%, about 1%, about 2%, about 5%, about 10%, about 15%, about 20%, about 25%, about 30%, about 35%, about 40%, about 50%, about 60%, about 70%, about 80%, or about 90% by weight of Compound 1. In certain embodiments, the pharmaceutical composition comprises about 0.25%, about 0.5% or about 1% by weight of Compound 1.

In certain embodiments, the pharmaceutical composition provided herein comprises from about 1 mg to about 5,000 mg, from about 10 mg to about 2,000 mg, from about 50 mg to about 1,000 mg, from about 100 mg to about 1,000 mg, or from about 100 mg to about 500 mg of Compound 1. In certain embodiments, the pharmaceutical composition provided herein comprises about 125 mg, about 200 mg, about 325 mg, about 400 mg, or about 500 mg of Compound 1. In certain embodiments, the pharmaceutical composition provided herein comprises from about 120 mg to about 130, from about 195 mg to about 205 mg, from about 320 mg to about 330 mg, from about 395 mg to about 405 mg, or from about 495 mg to about 505 mg of Compound 1.

In certain embodiments, Compound 1 in the pharmaceutical compositions provided herein is the free acid of 3-[5-(2-fluoro-phenyl)-[1,2,4]oxadiazol-3-yl]-benzoic acid or a salt of 3-[5-(2-fluoro-phenyl)-[1,2,4]oxadiazol-3-yl]-benzoic acid, as provided herein.

The pharmaceutical compositions provided herein can be provided in a unit dosage form or multiple-dosage form. A unit dosage form, as used herein, refers to a physically discrete unit suitable for administration to a human or animal subject using packaging known in the art. Each unit-dose contains a predetermined quantity of an active ingredient(s) sufficient to produce the desired therapeutic effect, in association with the required pharmaceutical carriers or excipients. Examples of a unit dosage form include, without limitation, an individually packaged packet, sachet or bottle or dropper. A unit dosage form may be administered in fractions or multiples thereof. A multiple-dosage form is a plurality of identical unit dosage forms packaged in a single container to be administered as segregated or combined unit dosage forms. Examples of a multiple-dosage form include a packet or sachet of granules or powder, a vial or bottle of tablets or capsules, or a bottle of liquid solution in fluid ounces, pints or gallons for administration either parenterally, orally or ocularly via dropper.

The pharmaceutical compositions provided herein can be administered as a divided dose over a period of time. It is understood that the precise dosage and duration of treatment may vary with the age, weight, and condition of the patient being treated, and may be determined empirically using known testing protocols or by extrapolation from in vivo or in vitro test or diagnostic data or by observation of certain clinical factors. It is further understood that for any particular individual, specific dosage regimens may be adjusted over time according to the individual need and the professional judgment of the person administering or supervising the administration of the pharmaceutical composition.

5.4.1. Topical Formulations

In certain embodiments, the pharmaceutical compositions provided herein comprise a micronized form of Compound 1 having enhanced solubility. In certain embodiments, the pharmaceutical compositions provided herein comprise a nanoparticle form of Compound 1 having enhanced solubility and/or dissolution rate.

In specific embodiments, the pharmaceutical compositions provided herein comprise a micronized form of Compound 1 wherein >90% of the particles of Compound 1 have a diameter (D₉₀ value) of between about 1-10 microns having enhanced solubility. In certain embodiments, the pharmaceutical compositions provided herein comprise a micronized form of Compound 1 a D₉₀ value of about 10 microns, about 9 microns, about 8 microns, about 7 microns, about 6 microns, about 5 microns, about 4 microns, about 3 microns, about 2 microns or about 1 micron having enhanced solubility. In certain embodiments, the pharmaceutical compositions provided herein comprise a micronized form of Compound 1 a D₉₀ value of between about 1-5 microns having enhanced solubility and/or dissolution rate. In certain embodiments, the pharmaceutical compositions provided herein comprise a micronized form of Compound 1 a D₉₀ value of about 5 microns, about 4 microns, about 3 microns, about 2 microns or about 1 micron having enhanced solubility and/or dissolution rate. In certain embodiments, the pharmaceutical compositions provided herein comprise a nanoparticle form of Compound 1 having enhanced solubility. In specific embodiments, the pharmaceutical compositions provided herein comprise a nanoparticle form of Compound 1 wherein >90% of the particles of Compound 1 have a D₉₀ value of about 0.1 microns, about 0.09 microns, about 0.08 microns, about 0.07 microns, about 0.06 microns, about 0.05 microns, about 0.04 microns, about 0.03 microns, about 0.02 microns or about 0.01 microns.

5.4.2. Oral Formulations

In certain embodiments, the pharmaceutical compositions provided herein are formulated for oral administration. In certain embodiments, the pharmaceutical compositions provided herein for oral administration are provided in solid, semisolid, or liquid dosage forms for oral administration. As used herein, oral administration also includes buccal, lingual, and sublingual administration. Suitable oral dosage forms include, but are not limited to, tablets, sublingual or buccal films (i.e., fastmelts), chewable tablets, effervescent tablets, mini-tablets, capsules, pills, strips, troches, lozenges, pastilles, cachets, pellets, medicated chewing gum, bulk powders or granules, effervescent or non-effervescent powders or granules, oral mists, solutions, emulsions, suspensions, wafers, sprinkles, elixirs, and syrups. In addition to the active ingredient, the pharmaceutical compositions can contain one or more pharmaceutically acceptable carriers or excipients including, but not limited to, binders, fillers, diluents, disintegrants, wetting agents, surfactants, lubricants, glidants, pH-modifiers, coloring agents, dye-migration inhibitors, sweetening agents, flavoring agents, emulsifying agents, suspending and dispersing agents, preservatives, solvents, solvating agents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose (CMC), carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methyl cellulose and carboxymethyl cellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include, but are not limited to, colloidal silicon dioxide, CAB-O-SIL® (Cabot Co. of Boston, Mass.), and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve a plurality of functions, even within the same formulation.

The pharmaceutical compositions provided herein as a tablet for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredient from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient.

The pharmaceutical compositions provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, solvating agent or emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, ethylenediamine tetraacetic acid (EDTA), hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration can be also provided in the forms of liposomes, micelles, microspheres, or nanosystems.

The pharmaceutical compositions provided herein for oral administration can be provided as either non-effervescent or effervescent tablets or granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

In certain embodiments, the pharmaceutical composition is formulated as a solid oral dosage form. In certain embodiments, the pharmaceutical composition is formulated as a liquid oral dosage form. In certain embodiments, the unit dosage form is provided as a suspension after being mixed in a pharmaceutically acceptable liquid or semi-solid solvating agent, which includes, but is not limited to, water, milk, carbonated beverage, juice, fruit juice, fruit punch, applesauce, yogurt, pudding, ice cream, baby food, baby formula or a soy or grain based product.

In certain embodiments, provided herein are pharmaceutical compositions, which comprise 3-[5-(2-fluoro-phenyl)-[1,2,4]oxadiazol-3-yl]benzoic acid or a pharmaceutically acceptable salt thereof and one or more additional pharmaceutically acceptable excipients. In one embodiment, the pharmaceutical composition is formulated as a powder. In one embodiment, the pharmaceutical composition is formulated as a micronized powder. In one embodiment, the pharmaceutical composition is formulated as a nanoparticle. In one embodiment, the pharmaceutical composition is formulated as granules. In another embodiment, the one or more excipients are selected from the group consisting of polydextrose, mannitol, poloxamer, polyethylene glycol, hydroxyethyl cellulose, crospovidone, artificial flavoring, and magnesium stearate. In certain embodiments, the artificial flavoring is an artificial vanilla flavor.

Additionally provided herein are pharmaceutical composition comprising about 25% by weight of 3-[5-(2-fluoro-phenyl)-[1,2,4]oxadiazol-3-yl]-benzoic acid or a pharmaceutically acceptable salt thereof; about 1% by weight of colloidal silicon dioxide; and one or more additional pharmaceutically acceptable excipients. In certain embodiments, pharmaceutical compositions provided herein comprise 3-[5-(2-fluoro-phenyl)-[1,2,4]oxadiazol-3-yl]-benzoic acid or a pharmaceutically acceptable salt thereof and one or more excipients selected from polydextrose, poloxamer (e.g., poloxamer 407), polyethylene glycol (e.g., polyethylene glycol 3350), mannitol, hydroxyethyl cellulose, artificial vanilla flavoring, crospovidone, colloidal silicon dioxide, and magnesium stearate (e.g., of vegetable origin). In certain embodiments, pharmaceutical compositions provided herein comprise 3-[5-(2-fluoro-phenyl)-[1,2,4]oxadiazol-3-yl]-benzoic acid or a pharmaceutically acceptable salt thereof and one or more excipients selected from a suspending agent, a binding agent that can also provide taste-masking, surfactant agent, a disintegrant and other excipients can be present. In one embodiment, the pharmaceutical composition is formulated as a powder. In one embodiment, the pharmaceutical composition is formulated as a micronized powder. In one embodiment, the pharmaceutical composition is formulated as a nanoparticle. In one embodiment, the pharmaceutical composition is formulated as granules. In another embodiment, 3-[5-(2-fluoro-phenyl)-[1,2,4]oxadiazol-3-yl]-benzoic acid or a pharmaceutically acceptable salt thereof is present in an amount such as at about 25% by weight). In another embodiment, the one or more excipients are selected from the group consisting of polydextrose, mannitol, poloxamer, polyethylene glycol, hydroxyethyl cellulose, crospovidone, artificial vanilla flavor, and magnesium stearate. In another embodiment, the one or more excipients (and their proportions of the total formulation weight) are selected from the group consisting of a suspending agent such as Litesse® Ultra [refined polydextrose] at about 26% by weight, a binding agent such as mannitol at about 26% by weight, surfactant agents such as polyethylene glycol 3350 at about 10.0% by weight and Lutrol® micro F127 [poloxamer 407 powder] at about 4% by weight, a disintegrant such as crospovidone at about 5% by weight, and other excipients, each less than about 2% by weight such as cab-o-sil, hydroxyethyl cellulose, magnesium stearate [non-bovine] at about 1% by weight and colloidal silicon dioxide at about 1% by weight.

Further provided herein are pharmaceutical compositions comprising about 25% by weight of 3-[5-(2-fluoro-phenyl)-[1,2,4]oxadiazol-3-yl]-benzoic acid or a pharmaceutically acceptable salt thereof, about 26% by weight of polydextrose, about 26% by weight of mannitol, about 3% by weight of poloxamer, about 10% by weight of polyethylene glycol, about 2% by weight of hydroxyethyl cellulose, about 5% by weight of crospovidone, about 1% by weight of artificial vanilla flavor, about 1% by weight of colloidal silicon dioxide and about 1% by weight of magnesium stearate. In one embodiment, the pharmaceutical composition is formulated as a powder. In one embodiment, the pharmaceutical composition is formulated as a micronized powder. In one embodiment, the pharmaceutical composition is formulated as a nanoparticle. In one embodiment, the pharmaceutical composition is formulated as granules.

Further provided herein are pharmaceutical compositions comprising, 3-[5-(2-fluoro-phenyl)-[1,2,4]oxadiazol-3-yl]-benzoic acid or a pharmaceutically acceptable salt thereof in a range of from about 120 mg to about 1005 mg, polydextrose in a range of from about 133 mg to about 1030 mg, mannitol in a range of from about 137 mg to about 901 mg, poloxamer in a range of from about 19 mg to about 147 mg, polyethylene glycol in a range of from about 52 mg to about 402 mg, hydroxyethyl cellulose in a range of from about 7 mg to about 59 mg, crospovidone in a range of from about 26 mg to about 201 mg, artificial vanilla flavor in a range of from about 3 mg to about 29 mg, colloidal silicon dioxide in a range of from about 5 mg to about 39 mg and magnesium stearate in a range of from about 5 mg to about 39 mg. In one embodiment, the pharmaceutical composition is formulated as powder. In one embodiment, the pharmaceutical composition is formulated as a powder. In one embodiment, the pharmaceutical composition is formulated as a micronized powder. In one embodiment, the pharmaceutical composition is formulated as a nanoparticle. In one embodiment, the pharmaceutical composition is formulated as granules.

Further provided herein are pharmaceutical compositions, comprising about 130 mg of 3-[5-(2-fluoro-phenyl)-[1,2,4]oxadiazol-3-yl]-benzoic acid or a pharmaceutically acceptable salt thereof, about 133 mg of polydextrose, about 137 mg of mannitol, about 19 mg of poloxamer, about 52 mg of polyethylene glycol, about 7 mg of hydroxyethyl cellulose, about 26 mg of crospovidone, about 3 mg of artificial vanilla flavor, about 5 mg of colloidal silicon dioxide and about 5 mg of magnesium stearate. In one embodiment, the pharmaceutical composition is formulated as a powder. In one embodiment, the pharmaceutical composition is formulated as a micronized powder. In one embodiment, the pharmaceutical composition is formulated as a nanoparticle. In one embodiment, the pharmaceutical composition is formulated as granules.

Further provided herein are pharmaceutical compositions, comprising about 205 mg of 3-[5-(2-fluoro-phenyl)-[1,2,4]oxadiazol-3-yl]-benzoic acid or a pharmaceutically acceptable salt thereof, about 210 mg of polydextrose, about 216 mg of mannitol, about 30 mg of poloxamer, about 82 mg of polyethylene glycol, about 12 mg of hydroxyethyl cellulose, about 41 mg of crospovidone, about 6 mg of artificial vanilla flavor, about 8 mg of colloidal silicon dioxide and about 8 mg of magnesium stearate. In one embodiment, the pharmaceutical composition is formulated as a powder. In one embodiment, the pharmaceutical composition is formulated as a micronized powder. In one embodiment, the pharmaceutical composition is formulated as a nanoparticle. In one embodiment, the pharmaceutical composition is formulated as granules.

Further provided herein are pharmaceutical compositions, comprising about 330 mg of 3-[5-(2-fluoro-phenyl)-[1,2,4]oxadiazol-3-yl]-benzoic acid or a pharmaceutically acceptable salt thereof, about 338 mg of polydextrose, about 348 mg of mannitol, about 48 mg of poloxamer, about 132 mg of polyethylene glycol, about 19 mg of hydroxyethyl cellulose, about 66 mg of crospovidone, about 9 mg of artificial vanilla flavor, about 13 mg of colloidal silicon dioxide and about 13 mg of magnesium stearate. In one embodiment, the pharmaceutical composition is formulated as a powder. In one embodiment, the pharmaceutical composition is formulated as a micronized powder. In one embodiment, the pharmaceutical composition is formulated as a nanoparticle. In one embodiment, the pharmaceutical composition is formulated as granules.

Further provided herein are pharmaceutical compositions, comprising about 405 mg of 3-[5-(2-fluoro-phenyl)-[1,2,4]oxadiazol-3-yl]-benzoic acid or a pharmaceutically acceptable salt thereof, about 415 mg of polydextrose, about 427 mg of mannitol, about 59 mg of poloxamer, about 162 mg of polyethylene glycol, about 24 mg of hydroxyethyl cellulose, about 81 mg of crospovidone, about 12 mg of artificial vanilla flavor, about 16 mg of colloidal silicon dioxide and about 16 mg of magnesium stearate. In one embodiment, the pharmaceutical composition is formulated as a powder. In one embodiment, the pharmaceutical composition is formulated as a micronized powder. In one embodiment, the pharmaceutical composition is formulated as a nanoparticle. In one embodiment, the pharmaceutical composition is formulated as granules.

Further provided herein are pharmaceutical compositions, comprising about 505 mg of 3-[5-(2-fluoro-phenyl)-[1,2,4]oxadiazol-3-yl]-benzoic acid or a pharmaceutically acceptable salt thereof, about 518 mg of polydextrose, about 453 mg of mannitol, about 74 mg of poloxamer, about 202 mg of polyethylene glycol, about 30 mg of hydroxyethyl cellulose, about 101 mg of crospovidone, about 15 mg of artificial vanilla flavor, about 20 mg of colloidal silicon dioxide and about 20 mg of magnesium stearate. In one embodiment, the pharmaceutical composition is formulated as a powder. In one embodiment, the pharmaceutical composition is formulated as a micronized powder. In one embodiment, the pharmaceutical composition is formulated as a nanoparticle. In one embodiment, the pharmaceutical composition is formulated as granules.

Further provided herein are pharmaceutical compositions, comprising about 1005 mg of 3-[5-(2-fluoro-phenyl)-[1,2,4]oxadiazol-3-yl]-benzoic acid or a pharmaceutically acceptable salt thereof, about 1030 mg of polydextrose, about 901 mg of mannitol, about 147 mg of poloxamer, about 402 mg of polyethylene glycol, about 59 mg of hydroxyethyl cellulose, about 201 mg of crospovidone, about 29 mg of artificial vanilla flavor, about 39 mg of colloidal silicon dioxide and about 39 mg of magnesium stearate. In one embodiment, the pharmaceutical composition is formulated as a powder. In one embodiment, the pharmaceutical composition is formulated as a micronized powder. In one embodiment, the pharmaceutical composition is formulated as a nanoparticle. In one embodiment, the pharmaceutical composition is formulated as granules.

In certain embodiments, the unit dosage form comprises from about 35 mg to about 5,600 mg of Compound 1, from about 35 mg to about 2800 mg of Compound 1, from about 35 mg to about 1,400 mg of Compound 1, from about 125 mg to about 1,000 mg of Compound 1, from about 250 mg to about 1,000 mg of Compound 1, from about 325 mg to about 1,000 mg of Compound 1 or from about 500 mg to about 1,000 mg of Compound 1.

In certain embodiments, the unit dosage form comprises about 35 mg, about 50 mg, about 70 mg, about 100 mg, about 125 mg, about 140 mg, about 175 mg, about 200 mg, about 250 mg, about 280 mg, about 325 mg, about 350 mg, about 400 mg, about 450 mg, about 500 mg, about 560 mg, about 700 mg, about 750 mg, about 1,000 mg, about 1,400 mg, about 2800 mg or about 5600 mg of Compound 1.

In certain embodiments, the pharmaceutical composition is formulated as a powder. In one embodiment, the pharmaceutical composition is formulated as a micronized powder. In one embodiment, the pharmaceutical composition is formulated as a nanoparticle. In another embodiment, the pharmaceutical composition provided herein is formulated as granules. In certain embodiments, the pharmaceutical composition provided herein is packaged in a packet or sachet. In certain embodiments, the pharmaceutical composition provided herein is packaged in a heat-sealed laminated aluminum packet or sachet. In certain embodiments, the pharmaceutical composition provided herein is packaged in a child-resistant packet or sachet. In certain embodiments, the pharmaceutical composition provided herein is packaged in a packet or sachet, which comprises layers of polyethylene terephthalate, polyethylene, aluminum foil, adhesive, and sealing film. In certain embodiments, the pharmaceutical composition provided herein is packaged in a bottle including, but not limited to, high density polyethylene (HDPE) bottles.

In certain embodiments, the pharmaceutical composition provided herein is formulated as granules for reconstitution. In certain embodiments, the pharmaceutical composition provided herein is formulated as granules for reconstitution as an oral suspension.

In certain embodiments, the pharmaceutical composition provided herein is reconstituted before administration by being mixed to a suspension with a pharmaceutically acceptable liquid or semi-solid solvating agent which includes, but is not limited to, water, milk, carbonated beverage, juice, fruit juice, fruit punch, applesauce, yogurt, pudding, ice cream, baby food, baby formula or a soy or grain based product.

In certain embodiments, the pharmaceutical composition provided herein is reconstituted before administration by being mixed to a suspension with water. In one embodiment, reconstitution of a 125 mg unit dosage formulation of Compound 1 is carried out by the addition of at least about 5 mL of water directly in a bottle containing Compound 1 to achieve a nominal concentration of at least about 25 mg/mL in the total volume of suspension. In another embodiment, reconstitution of a 250 mg unit dosage formulation Compound 1 is carried out by the addition of at least about 10 mL of water directly in a bottle containing Compound 1 to achieve a nominal concentration of at least about 25 mg/mL in the total volume of suspension. In another embodiment, reconstitution of a 500 mg unit dosage formulation Compound 1 is carried out by the addition of at least about 20 mL of water directly in a bottle containing Compound 1 to achieve a nominal concentration of at least about 25 mg/mL in the total volume of suspension. In another embodiment, reconstitution of a 1000 mg unit dosage formulation Compound 1 is carried out by the addition of at least about 40 mL of water directly in a bottle containing Compound 1 to achieve a nominal concentration of at least about 25 mg/mL in the total volume of suspension.

In other embodiments, a unit dosage form containing the pharmaceutical composition provided herein is only opened at the time of dose preparation. The full contents of each unit dosage form is mixed to a suspension with a liquid or a semi-solid solvating agent, wherein the liquid is at least 30 mL (1 ounce) or the semi-solid is at least 3 tablespoons. The prepared dose should be mixed well before being administered. The amount of the liquid or semi-solid solvating agent can be increased based on patient preference.

In certain embodiments, the pharmaceutical composition provided herein comprises Compound 1 as a free acid or as a pharmaceutically acceptable salt, wherein the pharmaceutically acceptable salt is a magnesium salt, a potassium salt, a sodium salt, a tromethamine salt, an L-lysine salt, an L-arginine salt, an N-methyl glucamine salt or an L-histidine salt.

5.4.3. Parenteral Formulations and Administration

The pharmaceutical compositions provided herein comprising Compound 1 can be administered parenterally by injection, infusion, or implantation, for local or systemic administration. Parenteral administration, as used herein include, without limitation, intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein for parenteral administration can be formulated in any dosage forms that are suitable for parenteral administration including, without limitation, solutions, suspensions, emulsions, micelles, liposomes, microspheres, nanosystems and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcellulose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

When the pharmaceutical compositions provided herein are formulated for multiple dosage administration, the multiple dosage parenteral formulations must contain an antimicrobial agent at bacteriostatic or fungistatic concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in the body, but allows the active ingredient in the pharmaceutical compositions to be osmotically or ionically diffused.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

5.4.4. Particle Size

Provided herein are micronized forms of Compound 1 having a volume weighted mean diameter D[4,3] of from about 2 μm to about 12 μm. Also provided herein are micronized forms of Compound 1 having a surface weighted mean diameter D[3,2] of from about 1 μm to about 3 μm. Further provided herein are forms of Compound 1 having a D₉₀ particle size in the range of from about 5 μm to about 26 μm, having a D₅₀ particle size in the range of from about 1 μm to about 6 μm, having a D₁₀ particle size in the range of from about 0.1 μm to about 1.5 μm.

5.4.5. Kits

The pharmaceutical compositions provided herein can be provided as an article of manufacture using packaging materials well known to those of skill in the art. Examples of pharmaceutical packaging materials include, but are not limited to, blister packs, bottles, packets, sachets, tubes, inhalers, pumps, bags, vials, containers, syringes, droppers, and any packaging material suitable for a selected formulation and intended mode of administration and treatment.

Provided herein are kits which, when used by the medical practitioner, can simplify the administration of appropriate amounts of the active ingredient to a subject. In certain embodiments, the kit provided herein includes a container and a dosage form of a pharmaceutical formulation provided herein and instructions for use thereof. In certain embodiments, the instructions included with the kit provide guidance with respect to the dosage amounts and/or dosing regimens for administration of Compound 1.

In certain embodiments, the kit includes a container comprising a dosage form of the pharmaceutical formulation provided herein, in a container comprising one or more other therapeutic agent(s) described herein.

Kits provided herein can further include devices that are used to administer the active ingredient. Examples of such devices include, but are not limited to, syringes, needle-less injectors, drip bags, patches, droppers and inhalers.

Kits provided herein can further include pharmaceutically acceptable vehicles that can be used to administer the active ingredient. For example, if the active ingredient is provided in a solid form that must be reconstituted for parenteral administration, the kit can comprise a sealed container of a suitable vehicle in which the active ingredient can be dissolved to form a particulate-free sterile solution that is suitable for parenteral administration or can be reconstituted as a suspension for oral administration. Examples of pharmaceutically acceptable vehicles include, but are not limited to: aqueous vehicles including, but not limited to, Water for Injection USP, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol, and polypropylene glycol; and non-aqueous vehicles including, but not limited to, corn oil, cottonseed oil, peanut oil, sesame oil, ethyl oleate, isopropyl myristate, and benzyl benzoate.

### 5.5. Methods of Use

Provided herein are methods for treating, preventing, ameliorating or managing a nonsense mutation mediated MPS disease, comprising administering a 1,2,4-oxadiazole benzoic acid to a patient having a nmMPS. In particular, provided herein are methods of treating, preventing, ameliorating or managing nmMPS I by modulation of premature translation termination or nonsense-mediated mRNA decay, comprising administering to a patient having a type of nmMPS capable of being ameliorated by modulation of premature translation termination or nonsense-mediated mRNA decay an effective amount of a pharmaceutical composition comprising 3-[5-(2-fluoro-phenyl)-[1,2,4]oxadiazol-3-yl]benzoic acid or a salt thereof and one or more pharmaceutically acceptable carriers and excipients, as provided herein. In one or more embodiments, the nmMPS is nonsense mutation mediated MPS I resulting from certain nonsense mutations on one or both alleles of the IDUA gene. In certain embodiments, nmMPS I results from a nonsense mutation on one or both alleles of the IDUA gene selected from Q60X, Y64X, Q70X, Y167X, Q310X, Q320X, Q400X, W402X, G409X, Y581X, R619X, R621X, R626X, R628X and the like.

Further provided herein are methods for treating, preventing, ameliorating or managing a symptom of nmMPS I by modulation of premature translation termination or nonsense-mediated mRNA decay, comprising administering to a patient having a symptom of a type of nmMPS I capable of being ameliorated by modulation of premature translation termination or nonsense-mediated mRNA decay an effective amount of a pharmaceutical composition comprising 3-[5-(2-fluoro-phenyl)-[1,2,4]oxadiazol-3-yl]benzoic acid or a salt thereof and one or more pharmaceutically acceptable carriers and excipients, as provided herein.

Further provided herein are methods for treating, preventing, ameliorating or managing a type of nmMPS I associated with a nonsense mutation or a premature stop codon, comprising administering to a patient having a type of nmMPS I associated with a nonsense mutation or a premature stop codon an effective amount of a pharmaceutical composition comprising 3-[5-(2-fluoro-phenyl)-[1,2,4]oxadiazol-3-yl]benzoic acid or a salt thereof and one or more pharmaceutically acceptable carriers and excipients, as provided herein. In certain embodiments, nmMPS I results from certain mutations on one or both alleles of the IDUA gene. In certain embodiments, nmMPS I results from a Q60X, Y64X, Q70X, Y167X, Q310X, Q320X, Q400X, W402X, G409X, Y581X, R619X, R621X, R626X, R628X and the like nonsense mutation on one or both alleles of the IDUA gene.

Further provided herein are methods for treating, preventing, ameliorating or managing a symptom of nmMPS I associated with a nonsense mutation or a premature stop codon, comprising administering to a patient having a symptom of a type of nmMPS I associated with a nonsense mutation or a premature stop codon an effective amount of a pharmaceutical composition comprising 3-[5-(2-fluoro-phenyl)-[1,2,4]oxadiazol-3-yl]benzoic acid or a salt thereof and one or more pharmaceutically acceptable carriers and excipients, as provided herein.

In certain embodiments, the methods provided herein comprise the administration of a pharmaceutical composition comprising 3-[5-(2-fluoro-phenyl)-[1,2,4]oxadiazol-3-yl]benzoic acid or a salt thereof and one or more pharmaceutically acceptable carriers and excipients, as provided herein, in three doses in a 24 hour period according to the formula: 1X, 1X, 2X, where X is a particular dose (e.g., 2 mg/kg, 5 mg/kg, 8 mg/kg, 10 mg/kg, 15 mg/kg or 20 mg/kg) of the active agent. In a specific embodiment, a pharmaceutical composition comprising 3-[5-(2-fluoro-phenyl)-[1,2,4]oxadiazol-3-yl]benzoic acid or a salt thereof and one or more pharmaceutically acceptable carriers and excipients, as provided herein, is continuously administered three times per 24 hour period for a plurality of 24 hour periods at doses of about 1 mg/kg to about 3 mg/kg (e.g., about 2 mg/kg), about 3 mg/kg to about 7 mg/kg (e.g., about 5 mg/kg), about 6 mg/kg to about 10 mg/kg (e.g., about 8 mg/kg), about 7 mg/kg to about 13 mg/kg (e.g., about 10 mg/kg), about 13 mg/kg to about 17 mg/kg (e.g., about 15 mg/kg), or about 18 mg/kg to about 22 mg/kg (e.g., about 20 mg/kg) of 3-[5-(2-fluoro-phenyl)-[1,2,4]oxadiazol-3-yl]benzoic acid or a salt thereof and one or more pharmaceutically acceptable carriers and excipients, as provided herein, for a plurality of 24 hour periods including, but not limited to, days, weeks, months or years. In another specific embodiment, a pharmaceutical composition comprising 3-[5-(2-fluoro-phenyl)-[1,2,4]oxadiazol-3-yl]benzoic acid or a salt thereof and one or more pharmaceutically acceptable carriers and excipients, as provided herein, is continuously administered three times per 24 hour period at doses of about 5 mg/kg to about 9 mg/kg (e.g., about 7 mg/kg), about 5 mg/kg to about 9 mg/kg (e.g., about 7 mg/kg) and 12 mg/kg to about 16 mg/kg (e.g., about 14 mg/kg) of the active agent for weeks, months or years. In a specific embodiment, a pharmaceutical composition comprising 3-[5-(2-fluoro-phenyl)-[1,2,4]oxadiazol-3-yl]benzoic acid or a salt thereof and one or more pharmaceutically acceptable carriers and excipients, as provided herein, is continuously administered three times per 24 hour period at doses of about 8 mg/kg to about 12 mg/kg (e.g., about 10 mg/kg), about 8 mg/kg to about 12 mg/kg (e.g., about 10 mg/kg) and about 18 mg/kg to about 22 mg/kg (e.g., about 20 mg/kg) of the active agent for days, weeks, months or years. In a specific embodiment, a pharmaceutical composition comprising 3-[5-(2-fluoro-phenyl)-[1,2,4]oxadiazol-3-yl]benzoic acid or a salt thereof and one or more pharmaceutically acceptable carriers and excipients, as provided herein, is continuously administered three times per 24 hour period at doses of about 18 mg/kg to about 22 mg/kg (e.g., about 20 mg/kg), about 18 mg/kg to about 22 mg/kg (e.g., about 20 mg/kg) and about 38 mg/kg to about 42 mg/kg (e.g., about 40 mg/kg) of the active agent for days, weeks, months or years. In each 24 hour period that the active agent is administered, it is preferably administered three times at approximately 6-, 6, and 12-hour intervals (e.g., at ˜7:00 AM after breakfast, ˜1:00 PM after lunch, and at ˜7:00 PM after supper).

Compound 1 therapeutic efficacy follows a bell-shaped dose-response curve, wherein achieving and maintaining a certain steady-state plasma concentration within a particular range results in maximal activity. Thus, it is believed that individual patients may require particular dosage amounts (i.e., 1X, 1X, 2X, where X is a particular dose such as 2 mg/kg, 5 mg/kg, 8 mg/kg, 10 mg/kg, 15 mg/kg or 20 mg/kg) in order to achieve a therapeutically effective plasma concentration of Compound 1.

In certain embodiment, the methods provided herein comprise maintaining a plasma concentration of Compound 1 of greater than: about 0.1 μg/mL, about 0.5 μg/mL, about 2 μg/mL, about 5 μg/mL, about 10 μg/mL, about 20 μg/mL, about 25 μg/mL, about 40 μg/mL, about 50 μg/mL, about 100 μg/mL, about 150 μg/mL, about 200 μg/mL, about 250 μg/mL or about 500 μg/mL in a patient for at least about 2, 2.5, 3, 3.5, 4, 4.5, 5, 6, 8, 12 or 24 hours or longer. Levels of Compound 1 in plasma can be measured, for example, by high performance liquid chromatography (HPLC).

In another embodiment, the methods provided herein comprise maintaining a plasma concentration of Compound 1 of about 0.1 μg/mL to about 500 μg/mL, about 2 μg/mL to about 40 μg/mL, about 2 μg/mL to about 20 μg/mL, about 2 μg/mL to about 10 μg/mL or about 10 μg/mL to about 20 μg/mL in a patient for at least about 2, 2.5, 3, 3.5, 4, 4.5, 5, 6, 8, 12 or 24 hours or longer.

In some embodiments, Compound 1 is administered in combination with at least one additional therapeutic agent. In some embodiments, the additional therapeutic agent is administered before administration of Compound 1, after administration of Compound 1, simultaneously with administration of Compound 1, or a combination thereof. In some embodiments, the additional therapeutic agent is laronidase (more specifically, α-L-iduronidase) and/or one or more anti-histamine(s).

In certain embodiments, the nonsense mutation mediated MPS disease to be treated, prevented, ameliorated or managed by the methods provided herein is nmMPS I. In certain embodiments, Compound 1 is used in combination with another therapy to treat nmMPS I. In a specific embodiment, the therapy used for the treatment, prevention, amelioration or management of nmMPS I in combination with Compound 1 or a pharmaceutical composition provided herein is enzyme replacement therapy (ERT), hematopoietic stem cell transplantation (HSCT), bone marrow transplantation (BMT) or umbilical cord blood transplantation (UCBT). In a specific embodiment, the therapy used for the treatment, prevention, amelioration or management of nmMPS I in combination with Compound 1 or a pharmaceutical composition provided herein is recombinant α-L-iduronidase, also known as Aldurazyme™ (brand of laronidase).

It will be understood that the amounts of a pharmaceutical composition or active agent administered to a patient in need thereof are or can be calculated based upon the actual weight of the patient in question or the average weight of the patient population in question (e.g., male or female, including adults and children).

### 6. EXAMPLES

6.1. Diagnosis of nmMPS I

Initial diagnosis is obtained using three biochemical tests. The first test assays GAG levels in urine. The second test chromatographically determines which GAG is present. The third test, based on which GAG is present, is an enzyme analysis to determine the level of enzymatic activity. The enzyme analysis is performed using plasma, leukocytes, or fibroblasts isolated from the patient. The enzyme assay can also determine the type of mucopolysaccharide disease, since patients with the severe form will have no enzymatic activity, whereas patients with the attenuated form will have reduced enzymatic activity of 0.1% or less. Determination of the genotype via DNA sequencing can determine the specific phenotype of nmMPS I. DNA sequencing can also confirm the diagnosis in patients where the biochemical results are ambiguous. Finally, genetic screening is used to determine future reproductive risks and to prevent a recurrence of nmMPS I in families that have affected children.

6.2. Effect of Compound 1 on GAG Levels in Cultured Fibroblasts from the Nonsense-Mutation Containing Hurler Syndrome (nmHurler) Mouse Model

nmMPS I is caused by the lack of the lysosomal enzyme IDUA and results in the accumulation of GAGs in tissues. Therefore, GAG levels can be monitored to assess the efficacy of treatment for this disease. In the nmHurler mouse model of nmMPS I, the Idua-W402X Hurler mouse IDUA gene (TGG→TAG) harbors a W392X mutation in exon 9, corresponding to the W402X mutation found in approximately 30 to 50% of nmMPS I patients. Mouse embryonic fibroblasts (MEFs) isolated from the Idua-W402X mice were treated with Compound 1 at concentrations of 0, 1, 2.5, 5, 10, 20, and 40 μg/mL for 24 hours in 0.2% DMSO. After 24 hours, cells were lysed and the levels of GAGs were quantified.

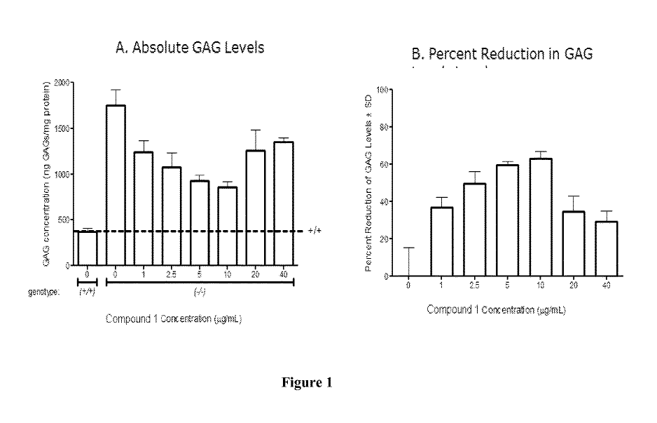

FIG. 1A shows GAG levels in MEFs from wild type mice, MEFs from Idua-W402X mice treated with vehicle, and MEFs from Idua-W402X mice treated with increasing concentrations of Compound 1. FIG. 1B shows the same data expressed as the reduction in excess tissue GAGs (calculated by subtracting the background GAG levels as measured in MEFs from wild type mice).

These data show that Compound 1 enabled readthrough of the premature stop codon present in the iduronidase mRNA, resulting in increased iduronidase activity in a concentration dependent manner, as determined by the dose-dependent changes of tissue GAGs. The greatest reduction of GAG levels occurred at 10 μg/mL with decreased reduction of GAG levels at concentrations above 20 μg/mL. No signs of cellular toxicity or stress (i.e., cells rounding up or floating, or changes in cell density) were observed up to the highest concentration tested (40 μg/mL). Cell viability was determined by quantifying the total protein concentration in each lysate. No notable changes in total protein concentration were observed with the Compound 1 concentrations tested, which was consistent with the lack of visual cytotoxicity (data not shown).

Mouse Model of nmMPS I

Compound 1 was administered to Idua-W402X mice by mixing Compound 1 into solid mouse chow at concentrations of 0.1%. 0.3% or 1.0% (w/w) for 2 weeks, after which time GAG levels in the brain, spleen, heart, liver, lung, and kidney tissues were quantified.

Maximal reduction of GAG levels in the brain, spleen, heart, and lung was obtained in animals treated with the 0.1% Compound 1 concentration (p<0.05, Student's t-test, Compound 1 treated vs vehicle), with near normal GAG levels achieved in the brain and spleen (Table 1). Maximal reduction in liver GAG levels was obtained in animals treated with 0.3% Compound 1 concentration (p<0.05, Student's t-test, Compound 1 treated vs vehicle). Increasing the Compound 1 concentrations above these levels led to less reduction of tissue GAG level. No statistically significant effect was observed in the kidney at any dose.

TABLE 1
Treatment with Compound 1 Reduces Tissue GAG Levels

| % | Reduction in Excess GAG Levels | | | | | |
|---|---|---|---|---|---|---|
| Cpd 1 | Brain | Spleen | Heart | Liver | Lung | Kidney |
| 0.1% | 70% | 78% | 36% | 0% | 19% | 0% |
| 0.3% | 37% | 57% | 0% | 53% | 11% | 0% |
| 1.0% | 28% | 11% | 0% | 17% | 14% | 0% |

6.3. Pharmacokinetics of Compound 1

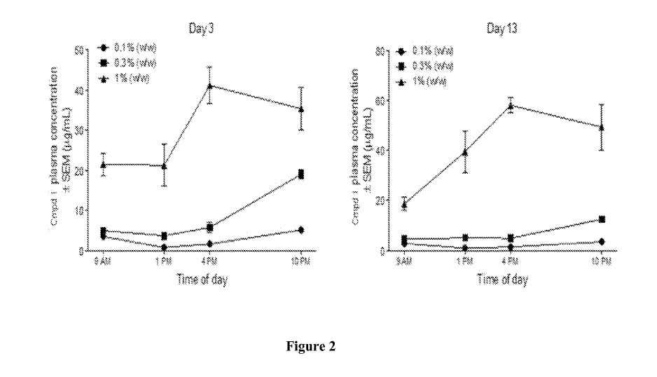

The pharmacokinetics of Compound 1 at three doses (0.1%, 0.3% or 1% (w/w)) when delivered via solid chow was determined in satellite wild type C57BL/6J mice. The first day Compound 1 was provided was defined as Day 0. On Day 3 and on Day 13 blood was obtained from mice at specified time points and the plasma concentration of Compound 1 was measured using liquid chromatography-tandem mass spectrometry. Compound 1 plasma concentrations increased with increasing dose as shown in FIG. 2 and summarized in Table 2.

C57BL/6J mice administered 1.0% (w/w) Compound 1 did not gain as much weight as mice administered 0.1%, 0.3% doses of Compound 1 or vehicle in both the satellite PK study as well as in the pharmacology studies using the Idua-W402X mice. Mice receiving food containing 1.0% (w/w) Compound 1 are 45% less food than mice receiving the control chow. However, a reduction in weight gain would not be expected to elicit a reduction in tissue GAG levels in the pharmacology studies.

Compound 1 doses of 0.1% and 0.3% (w/w) were associated with better efficacy in the nmHurler mouse model than a dose of 1.0% (w/w). From mice (n=4) at each time point, plasma concentrations ranged from 0.8 to 5.2 μg/mL at a dose of 0.1% (w/w) and from 3.7 to 19 μg/mL at a dose of 0.3% (w/w). Plasma concentrations ranged from 19-58 μg/mL at the 1.0% (w/w) dose.

TABLE 2
Compound 1 Plasma Exposure in Mice Dosed Via Chow (0.1% to 1.0%
(w/w))

| | Day 3 | | | Day 13 | | |
|---|---|---|---|---|---|---|
| % Cpd 1 | Cₘₐₓ (Tₘₐₓ) | Cₘᵢₙ (Tₘᵢₙ) | AUC₉₋₁₀ | Cₘₐₓ (Tₘₐₓ) | Cₘᵢₙ (Tₘᵢₙ) | AUC₉₋₁₀ |
| 0.1% | 5.2 (10 pm) | 0.76 (1 pm) | 35 | 3.6 (10 pm) | 1.0 (1 pm) | 28 |
| 0.3% | 19 (10 pm) | 3.7 (1 pm) | 109 | 13 (10 pm) | 4.6 (9 am) | 89 |
| 1.0% | 41 (4 pm) | 21 (1 pm) | 420 | 58 (4 pm) | 19 (9 am) | 594 |

AUC₉₋₁₀ refers to area under the curve plotting plasma concentration over time (9 am to 10 pm) (hr·μg/mL); Cₘₐₓ refers to maximal plasma concentration (μg/mL); Cₘᵢₙ refers to minimal plasma concentration (μg/mL); Tₘₐₓ refers to the time at which the maximal plasma concentration was measured; and Tₘᵢₙ refers to the time at which the minimal plasma concentration was measured.

6.4. Effect of Compound 1 on Iduronidase Activity in Cultured Fibroblasts from the nmHurler Mouse Model

Objective:

To determine the activity of Compound 1 in primary fibroblasts isolated from Idua-W392X mouse embryos.

Methods and Results:

The effect of Compound 1 on iduronidase activity as a result of readthrough of the premature stop codon present in the Idua gene was determined in an in vitro GAG accumulation assay. This assay utilized primary fibroblasts isolated from 13 day-old +/+ and −/− Idua-W392X mouse embryos. Mouse embryonic fibroblasts (MEFs) were isolated from 13 day-old wild type and homozygous Idua-W392X mouse embryo littermates. The cells were grown in Dulbecco's Modified Eagle Media (DMEM) containing 10% bovine calf serum (BCS)+1% non-essential amino acids+1% penicillin/streptomycin at 37° C. and 5% CO₂. The MEFs were isolated and grown on collagen-coated plates until ˜50% confluent. MEFs from Idua-W392X mice were treated with increasing concentrations of Compound 1 dissolved in DMSO to a final concentration of 0.2%. Cells were incubated with Compound 1 for 24 hours. Controls included untreated wild type and untreated Idua-W392X MEFs. After 24 hours, cells were visually inspected for signs of cytotoxicity and cell stress and then lysed using M-Per Protein Reagent (Pierce) containing protease inhibitors (Complete-mini; Roche) per the manufacturer's instructions. The supernatant was used to determine glycosaminoglycan (GAG) levels, as described below.

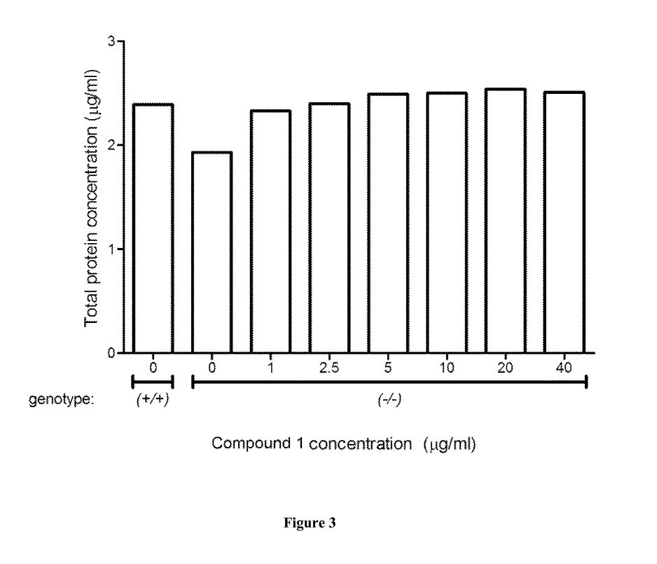

No signs of cell toxicity or stress were observed (i.e., cells rounding up or floating, changes in cell density) up to the highest concentration tested (40 ug/mL). The total protein concentration in each lysate was determined using the Bio-Rad protein assay with a standard curve generated using bovine serum albumin. No notable changes in total protein were observed over the concentrations tested (FIG. 3), consistent with the lack of visual cytotoxicity.

GAG levels were determined using the Blyscan Sulfated GAG Assay (Biocolor Ltd, UK). Briefly, 50 μl of each lysate was added to 500 μl of the Blyscan Dye Reagent and mixed by vortexing. The mixture was incubated at room temperature for 30 minutes, vortexing every 5 minutes. The dye-bound GAGs were isolated from the solution by centrifuging for 10 minutes at 10,000 g at room temperature. 500 μl of the Blyscan Dye Dissociation Reagent was added to the supernatant from each sample. The samples were vortexed and incubated for 10 minute at room temperature. The entire volume of each sample was then placed into a cuvette and the absorbance was measured at a wavelength of 656 nm. The total amount of sulfated GAGs precipitated from each sample was determined from a standard curve using chondroitin 4-sulfate. The total protein concentration in each lysate was determined using the Bio-Rad protein assay with a standard curve generated using bovine serum albumin.

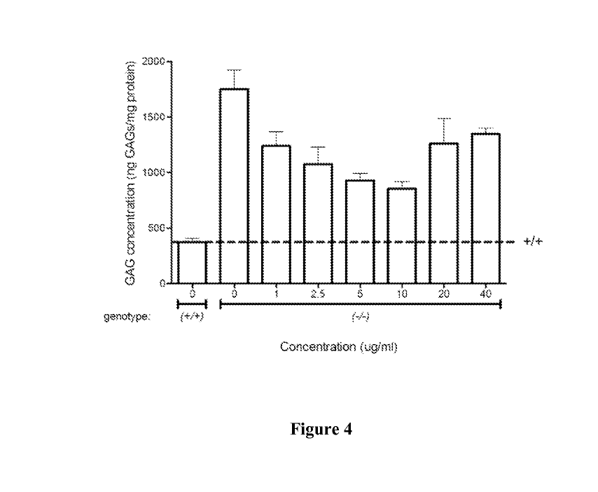

As shown in FIG. 4, Compound 1 exhibited a U shaped dose-response relationship for GAG reduction. The maximum level of GAG reduction was observed at 10 μg/mL.

Results:

The data presented here demonstrated that Compound 1 exhibited concentration dependent nonsense suppression activity as determined by GAG levels, and at higher concentrations, activity was reduced.

6.5. Effect of Compound 1 on Iduronidase Activity in the Nonsense-Containing Hurler Mouse Model

Objective:

To determine the activity of Compound 1 in the nonsense-mutation containing Idua-W392X Hurler mouse model.

Methods and Results:

The effect of Compound 1 on iduronidase activity as a result of readthrough of the premature stop codon present in the Idua gene (Idua-W392X) was determined in a nonsense-mutation containing mouse model of Hurler syndrome, referred to as the Idua-W392X Hurler mouse. The study initiated when mice were 3-4 weeks old.

Homozygous Idua-W392X mice were offered food prepared with or without Compound 1 for 14 days. Wild type animals received chow without Compound 1 for 14 days. Assay results were obtained from tissues isolated from wild type (+/+) and homozygous Idua-W392X (−/−) mice after 14 days of treatment. The concentrations of Compound 1 prepared in a chow formulation are shown in Table 1. Briefly, Compound 1 was dissolved in water, mixed into pulverized chow, shaped into pellets and dried.

TABLE 3
Compound 1 dose groups

| % Cpd 1^{a} | 0% | 0.1% | 0.3% | 1% |
|---|---|---|---|---|
| Wild-type | 6 | | | |
| Idua-W392X | 5 | 6 | 6 | 5 |

^{a}Purina 5001 chow

Following the 14-day treatment period, mice were anesthetized using isoflurane and transcardially perfused with 20 mL saline prior to tissue collection. At the time of sacrifice, brain, liver, heart, lung, spleen and kidney were collected and frozen at −80° C. until used for determination of GAG levels.

GAG levels were determined using the Blyscan Sulfated GAG Assay (Biocolor Ltd, UK). Briefly, 50 μL of each lysate was added to 500 μL of the Blyscan Dye Reagent and mixed by vortexing. The mixture was incubated at room temperature for 30 minutes, vortexing every 5 minutes. The dye-bound GAGs were isolated from the solution by centrifuging for 10 minutes at 10,000 g at room temperature. A total of 500 μL of the Blyscan Dye Dissociation Reagent was added to the supernatant from each sample. The samples were vortexed and incubated for 10 minute at room temperature. The entire volume of each sample was then placed into a cuvette and the absorbance was measured at a wavelength of 656 nm. The total amount of sulfated GAGs precipitated from each sample was determined from a standard curve using chondroitin 4-sulfate. The total protein concentration in each lysate was determined using the Bio-Rad protein assay with a standard curve generated using bovine serum albumin.

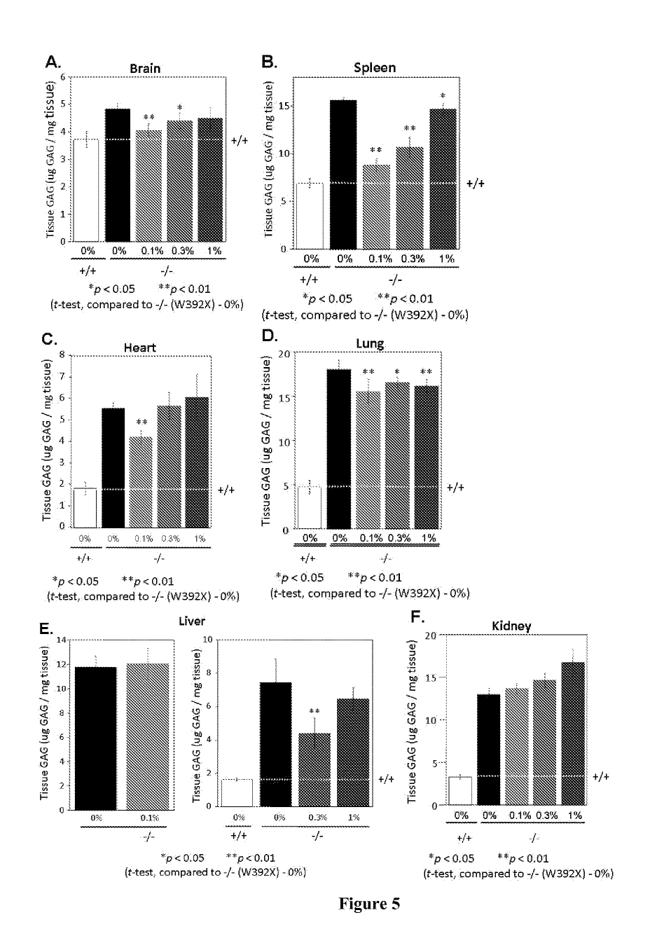

As shown in FIG. 5, lower concentrations of Compound 1 exhibited a greater response for several of the tissues where GAG reduction was observed (U shaped curve). The maximum level of GAG reduction was observed in the brain (FIG. 5A), spleen (FIG. 5B), heart (FIG. 5C), and lung (FIG. 5D) at 0.1% food and in the liver (FIG. 5E) at 0.3%. No statistically significant effect was observed in the kidney (FIG. 5F).

Results:

The data demonstrate that Compound 1 exhibited concentration-dependent nonsense suppression activity resulting in production of functional iduronidase as determined by decreased GAG levels in multiple tissues. At the lowest concentration evaluated (0.1% Compound 1), the maximum level of GAG reduction was observed in the brain, spleen, heart and lung. In the liver, the maximum effect was observed in the 0.3% Compound 1 dose group. No statistically significant effect was observed in the kidney at any dose administered. In those tissues where a response was observed, higher concentrations of Compound 1 resulted in reduced activity.

It will be appreciated that, although specific embodiments of the invention have been described herein for purposes of illustration, the invention described herein is not to be limited in scope by the specific embodiments herein disclosed. These embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description, which modification also intended to be within the scope of this invention.

What is claimed is:

1. A method for treating, preventing, ameliorating or managing a nonsense mutation mediated mucopolysaccharidosis (nmMPS) disease associated with a nonsense mutation or a premature stop codon, comprising administering an effective amount of 3-[5-(2-fluoro-phenyl)-[1,2,4]oxadiazol-3-yl]benzoic acid or a pharmaceutically acceptable salt thereof to a patient having the nmMPS disease.

2. A method for treating, preventing, ameliorating or managing nmMPS I associated with a nonsense mutation or a premature stop codon, comprising administering an effective amount of 3-[5-(2-fluoro-phenyl)-[1,2,4]oxadiazol-3-yl]benzoic acid or a pharmaceutically acceptable salt thereof to a patient having nmMPS I associated with a nonsense mutation or a premature stop codon.

3. The method of either of claim 1 or 2, wherein the salt is selected from the group consisting of a magnesium salt, a potassium salt, a sodium salt, a tromethamine salt, an L-lysine salt, an L-arginine salt, an N-methyl glucamine salt and an L-histidine salt.
